(19) United States
(12) Patent Application Publication
(10) Pub. No.: US 2007/0060924 A1
(43) Pub. Date: Mar. 15, 2007

(54) Recovery device for balloon kyphoplasty

(75) Inventor: Gil-Woon Choi, Incheon-shi (KR)

Correspondence Address: MATHEWS, SHEPHERD, MCKAY, & BRUNEAU, P.A., 29 THANET ROAD, SUITE 201, PRINCETON, NJ, 08540, US

(73) Assignee: Taeyon Medical Co., Ltd.

(21) Appl. No.: 11/508,381

(22) Filed: Aug. 23, 2006

(30) Foreign Application Priority Data
Aug. 29, 2005 (KR) 10-2005-0079518

(51) Int. Cl. A61F 2/00

(52) U.S. Cl. 606/93

(57) ABSTRACT

A recovery device for balloon kyphoplasty is disclosed, in which the entire weight, volume and size of the recovery device and manufacture cost can be decreased in such a manner that a box type synthetic resin body is adapted, the body being designed to properly hold a minimum portion of a balloon, a cylinder and a compressor. In addition, it is possible to manufacture a compact sized product.

### TECHNICAL FIELD

The present invention relates to a recovery device for a balloon kyphoplasty, and in particular to a disposable type balloon recovery device for a balloon kyphoplasty in which an entire construction of a recovery device may be simple by adapting a box type body which is designed to hold a balloon, a cylinder and a compressor. An excellent gripping and handling operation may be obtained, so that it is very easy to use the recovery device. With a pressure gauge having a LCD lamp display and an alarm generation device, an excellent visibility and safety operation are obtained, so that it is possible to prevent a medical accident.

### BACKGROUND ART

Generally, a balloon kyphoplasty for medically treating osteoporotic fractures of an aged person is a new significant treatment method since it is known to fully overcome any side effects of a conventional spine surgery method in which cement is forcibly injected without using balloon.

In a conventional spine surgery operation, since bone cement is forcibly injected into the interior of fallen spines without a separate space, an inner pressure of spines increases, so that bone cement liquid flows outside for thereby causing a big problem at neural systems.

High temperature may occur while bone cement liquid is hardened, so that surrounding neuron systems may be damaged by heat. Small particles of bone cement may be inputted into surrounding blood vessels while cement liquid is injected by over pressure. In this case, thrombosis may occur.

Therefore, the balloon spine surgery operation is capable of nearly overcoming the above side effects and is capable of recovering the spines bent due to osteoporotic fractures, so that it is possible to basically treat lumbago.

The operation method of the balloon spine surgery operation will be described as follows.

A thin needle is injected into a fallen spine in partial anesthesia, and balloon is expanded by an external strong air pressure(or pressure of liquid injected), so that an air pocket is made in the spines.

The thusly-manufactured air pocket balloon is very strongly expanded, so that it is possible to recover the fallen spines. It is checked whether the fallen spines are normally recovered to the original state or not by an X-ray test, and the air pocket is removed, and liquid bone cement is stably injected into the spines which have been recovered to the normal state for thereby finishing operation.

The recovery device, which is provided as an object of the present invention, is a certain surgical operation tool capable of recovering the damaged spines to an original state using a strong air pressure(or pressure of liquid injected).

The conventional recovery device comprises a balloon and a balloon body which are directly inserted into a spine, a cylinder which is connected with the balloon body through a tube, and a pusher which is assembled to a rear end of the cylinder for a pressurizing operation. The balloon is expanded by pressurizing the pusher based on two steps after the balloon and the balloon body are inserted into the spine.

However, in the case of the conventional recovery device, the balloon, balloon body, cylinder, pusher, etc. are formed in an integral construction, and the total number of parts is great, and it is not easy to handle the conventional recovery device.

In addition, since it is needed to push the pusher while holding the body portion of the cylinder when using the same, a gripping operation is poor, so that handling the conventional recovery device is not easy.

Since it is needed to adjust a proper pressure while checking the scales of the pressure gauge when pressurizing, a visibility decreases. According to the situation, a proper pressure may be exceeded, so that a medical accident may occur in the conventional art.

### DISCLOSURE OF THE INVENTION

Accordingly, it is an object of the present invention to provide a recovery device for balloon kyphoplasty which overcomes the problems encountered in the conventional art.

It is another object of the present invention to provide a recovery device for balloon kyphoplasty in which the entire weight, volume and size of the recovery device and manufacture cost can be decreased in such a manner that a box type synthetic resin body is adapted, the body being designed to properly hold a minimum portion of a balloon, a cylinder and a compressor. In addition, it is possible to manufacture a compact sized product.

It is further object of the present invention to provide a disposable type recovery device in which a compression release and a locking and unlocking of a pusher are possible by a simple operation of a lever by adapting an elastic lever type locking unit, so that a gripping operation is easy, and handling a recovery device is easy for thereby implementing a comfortable operation.

It is still further another object of the present invention to provide a disposable type recovery device in which there are provided a LCD lamp display at a pressure gauge for an easier check, and an alarm generation unit for generating an alarm signal when a certain pressure is exceeded, so that it is possible to obtain a good visibility for thereby preventing a medical accident.

To achieve the above objects, in a recovery device for a balloon kyphoplasty which includes a balloon and a balloon body directly inserted into a spine, a cylinder connected with the balloon body using a tube, and a pusher which is assembled at a rear end of the cylinder for thereby performing a pressurizing operation, there is provided a disposable type recovery device for a balloon kyphoplasty which comprises a box shape body which is integrally assembled at a rear end of the cylinder and partially receives a front end of the pusher for thereby selectively screw-engaging or disengaging the pusher using a pusher locking part, with an intermediate portion of the body being combined with two tube members which are separable in a longitudinal direction, and with the box shape body comprising a cylinder mounting part of a front side for receiving a rear end portion of the cylinder, and a hole structure block mounting part being disposed at a rear side of the cylinder mounting part with a partition being disposed between the same, and with the two tube members being combined with each other and being integrally assembled by outer rings 16a and 16b engaged at the step portions of the front and rear ends.

The pusher locking unit is provided with a locking block which includes a lever which is installed at an outer side of the body and is supported in a pin structure while being elastically supported by a spring and operates like a seesaw; and a screw part of which one side is pin-engaged at a rear end of the lever and which is movable up and down in the block mounting part and has an inner diameter portion engage with the pusher.

The body further includes a pressure gauge provided in the cylinder for measuring pressure, and the pressure gauge is designed to display a pressure data in a LED lamp display method and includes a buzzer for generating an alarm sound when a certain pressure level is reached when a balloon is expanded.

The cylinder and the pusher are integrally assembled in such a manner that a front end of the pusher is partially straight inserted into a pad of the cylinder, and an engaging pin passes through the inserted portion of the pad.

The disposable recovery device according to the present invention is formed in a compact size structure in which a cubic box body, namely, a synthetic resin box type body is integrally assembled at a connection potion of a cylinder and a pusher, with the body being easily movable with only one hand.

So, an operator can easily hold a body with one hand and handle the same while pressurizing a pusher with the other hand, so that it is very convenient to perform a surgical operation. With the above convenience, a safe and efficient surgical operation can be performed.

A lever is provided at one side of the body. It is very easy to handle the lever with a finger(for example, thumb). The compression releasing operation can be easily performed after the surgical operation is finished.

The above body is formed of a combination type member of two synthetic resin materials assembled by two outer rings engaged at the front and rear sides, the cylinder can be easily assembled in a state that the body is separated, and the present invention is a disposable type, the manufacture cost can be decreased.

The recovery device according to the present invention does not adapt a conventional complicated engagement between a cylinder and a pusher. Namely, in the present invention, it is possible to significantly decrease the number of parts by engaging a cylinder and a pusher using a simple pin engagement structure, so that the manufacture cost can be decreased.

In particular, the recovery device according to the present invention adapts a pressure gauge of a LCD lamp display type for an easier recognition. A certain alarm unit is provided for generating an alarm sound when pressure exceeds a set level, so that a proper pressure level can be accurately adjusted during a surgical operation, whereby it is possible to prevent a certain medical accident which may occur when pressure exceeds a certain set level. With the above advantageous effects, it is possible to minimize any burden of a patient, and balloon kyphoplasty operation can be stably performed.

### BRIEF DESCRIPTION OF THE DRAWINGS

The present invention will become better understood with reference to the accompanying drawings which are given only by way of illustration and thus are not limitative of the present invention, wherein;

FIG. 1 is a front cross sectional view illustrating a structure of a recovery device according to the present invention;

FIG. 2 is a plane cross sectional view illustrating a structure of a recovery device according to the present invention;

FIG. 3 is a cross sectional view taken along line A-A of FIG. 1; and

FIGS. 4A through 4D are cross sectional views illustrating a use state of a recovery device according to the present invention.

### MODES FOR CARRYING OUT THE INVENTION

The preferred embodiments of a disposable type recovery device according to the present invention will be described with reference to the accompanying drawings.

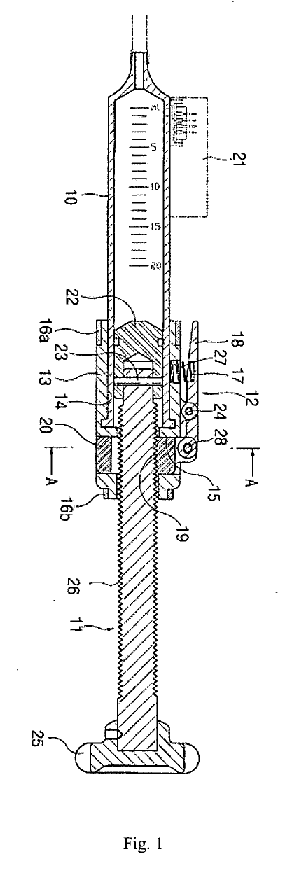

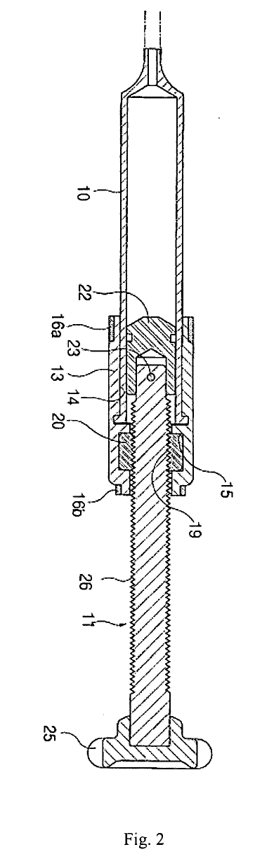

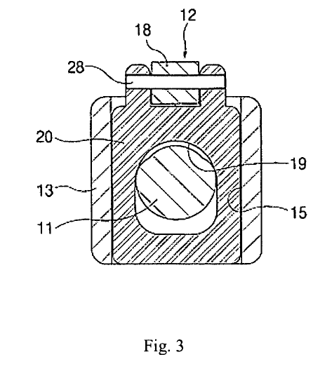

As shown in FIGS. 1 through 3, a recovery device according to the present invention comprises a balloon and a balloon body, a cylinder 10, and a pusher 11. There is further provided a body 13 for holding the entire structures of the cylinder 10 and the pusher 11.

The body 13 is formed in a rectangular box shape and comprises a cylinder mounting part 14 for receiving a rear side of the cylinder 10, and a block mounting part 15 for receiving a locking block 20 of a pusher locking part 12, with the cylinder mounting part 14 and the locking block 20 being provided at the front and rear sides in parallel and being provided at the inner side of the body 13, and with a pusher locking part 12 being provided at an outer side of the same for a compression release.

The body 13 is formed in a combination of two symmetrical tube members, and two tube members are combined in a width wise direction and are assembled in an integrated structure by outer rings 16a and 16b engaged at the step portions of the front and rear sides.

The thusly assembled bodies 13 may be separated while the intermediate portion of the width is divided in the longitudinal direction as the outer rings 16a and 16b are removed. If necessary, it may be separated from the cylinder 10 and the pusher 10.

The cylinder mounting part 14 is provided for receiving the cylinder 10, with an engaging groove being formed at an inner end portion of the same. The cylinder mounting part 14 is designed to receive the cylinder 10 in a state that the engaging end provided at a rear end of the cylinder remains caught, so that it is possible to prevent the cylinder mounting part 14 from being escaped in the direction of the front side of the cylinder 10.

The block mounting part 15 is provided for receiving the locking block 20, with the upper and lower sides of the same being opened. The block mounting part 15 is provided at the front and rear sides, respectively, in parallel about the partition with respect to the cylinder mounting part 14. A hole is formed at the partition and a wall body and concurrently passes through the same in a horizontal direction. With the above construction, the pusher 11, which is engaged at the cylinder 10o and is extended in the backward direction, passes through the hole and is longitudinally extended in the outer direction.

In the present invention, it is possible to more easily perform a backward movement of the pusher 11 for an operation release of the compressor, namely, a compression release. There is further provided a pusher locking part 12 which is screw-powered along with the pusher 11 for thereby performing an accurate pressurizing operation.

The body 13 is provided with the pusher locking part 12 formed of a lever 18 and a locking block 20.

In the pusher locking part 12, the lever 18 of which intermediate portion is supported by a hinge pin 24 is engaged at an upper outer portion of the body 13, and the lever 18 is elastically supported by a spring 17 disposed in a spring mounting groove 27 formed in the body 13. An upper end of the locking block 20 is connected at a rear end of the lever 18 by a connection pin 28, with the locking block 20 being movable up and down in the block mounting part 15.

Here, the locking block 20 is provided with a screw part 19 at its inner diameter portion, with the screw part being selectively engaged or disengaged from a screw bar 26 of the pusher 11 and operates in sync with the lever 18 which works about the hinge pin 24 like a seesaw for thereby engaging or disengaging the pusher 11.

For example, the lever 18 is elastically supported by the spring 17 supported by the body 13 and operates about a hinge pin 24 provided at an intermediate portion of the length like a seesaw. The locking block 20 is engaged at a rear end of the lever 18 by the connection pin 28 and is arranged in the interior of the block mounting part 15 formed in the body 13 and moves up and down based on the operation of the lever 18. Here, the locking block 20 has a hole which horizontally passes through the thickness wise portion and a screw part 19 formed at a semicircular section of an upper side of an inner diameter surface of the hole. The hole formed at the locking block 20 is nearly matched with the hole, which passes through the block mounting part 15 of the body 13. The hole formed at the locking block 20 is formed in a shape of an upper and lower elongated hole having a relatively large diameter, so that it is engaged or disengaged from the pusher 11 based on an up and down movement.

Namely, in a state that the lever 18 is not pressed, the rear end of the lever 18 is pulled in a downward direction by the force of the spring 17, and the locking block 20 is positioned at the lower side, so that the screw part 19 is engaged with the screw bar 26 provided at the pusher 11. When the lever is pressed, the engaged screw part 19 and screw bar 26 are disengaged, so that the restricting force of the pusher 11 is released.

When the handle 25 of the pusher 11 is handled in a state that the screw part 19 and the screw bar 26 are engaged with each other, the pusher 11 is slightly moved forward(as far as the pitch of the screw) for thereby pressurizing the cylinder side.

In the present invention, it is possible to more comfortably perform a surgical operation since the compression release can be performed by pressing the lever when the compression release is needed after the balloon expansion is finished.

The pusher 11 comprises a screw bar 26 having a handle 25 at a rear end, with a front end of the pusher 11 being connected with the side of the cylinder using the connection pin 28, so that the pusher is movable forward or backward.

The pusher 11 is partially straight-inserted into the pad 22 of the cylinder 10, and an engaging pin 23 passes through the upper side of the insertion part in the radial direction. The pusher 11 is integrally engaged with the side of the cylinder, so that the cylinder can be pressurized as the pusher 11 moves forwards and backward.

In the present invention, the number of parts can be decreased by adapting the engagement between the pusher 11 and the cylinder as compared to the conventional construction which includes knob, locking lever, spring, etc. In addition, the entire construction can be simplified.

The body 13 is provided with a pressure gauge 21 for measuring the pressure of the inner space of the cylinder which will be applied to the balloon.

The pressure gauge 21 is designed to display the pressure data by using the LED lamps. A buzzer is further provided so as to generate an alarm when the balloon reaches a certain pressure level when the balloon is expanded. Namely, when the pressure of the balloon exceeds a certain level, an alarm sound is generated, so that it is possible to prevent a certain medical accident.

In particular, the display window of the pressure gauge 21 is designed to display the pressure data in the LED lamp type for thereby significantly enhancing visibility.

The followings are the examples of the controls related with the operations of the pressure gauge 21.
1) RAM clear and register initialization
2) Input and output port setting
3) Input of pressure sensor converts into A/D
4) Set pressure is checked
5) Kg, bar switches are checked (kg display with one push, bar display with two pushes)
6) Buzzer generation when a set pressure reaches
7) Display windows flashes
8) Kg data are displayed on display window
9) Bar data are displayed on display window

The above control methods related with the pressure gauge may be implemented by known methods, and such adaptations may not be limited.

The operation of the recovery device according to the present invention will be described as follows.

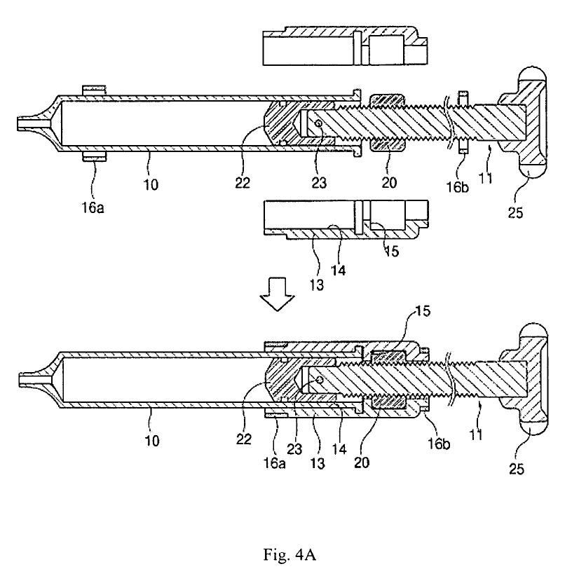

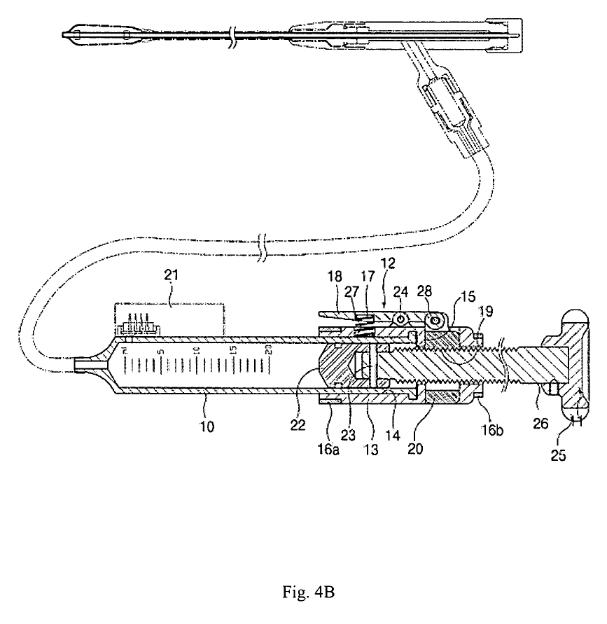

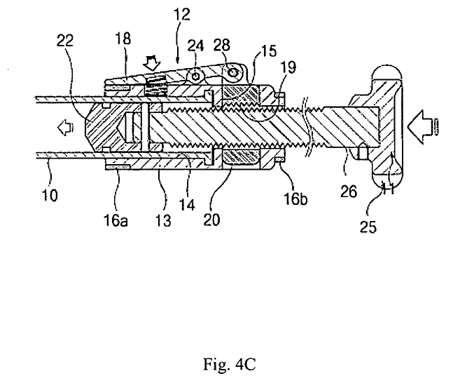

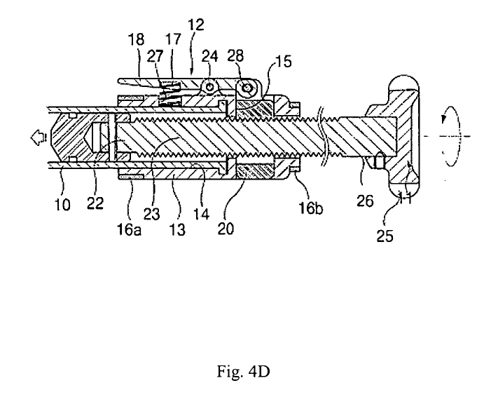

As shown in FIGS. 4A and 4B, the cylinder with the balloon, balloon body and tube, the pusher 11 and the body are assembled.

The body 12, which is divided into two members, is combined with the rear end of the cylinder 10 being disposed between the same. The outer rings 16a and 16b are engaged at the front and rear sides, so that the cylinder 10, the pusher 11 and the body 13 are integrally assembled.

The engaging end formed at the rear side of the cylinder 10 is inserted into the engaging groove of the inner side of the cylinder mounting part 11, so that the cylinder 10 is stably fixed at the body 14 while not being escaped in the forward direction.

The pusher 11 passes through the locking block 20 and is assembled thereto before the body 12 is assembled. Here, the pusher 11 may be assembled by passing the pusher 11 through the hole of the locking block 20 through the rear end of the screw bar 26. The handle 25 may be engaged at the rear end of the screw bar 26 using setscrews.

The assembling sequence of the above body, cylinder, pusher, etc. is not limited to the above-described sequence. A certain known assembling sequence may be adapted without any limitations.

When the assembling of the cylinder 10, the pusher 11 and the body 13 is finished, a spine recovery surgical operation is performed.

For example, a thin needle is injected into a fallen spine in partial anesthesia, and balloon is expanded by an external strong air pressure(or pressure of liquid injected), so that an air pocket is made in the spines.

When the balloon is expanded, it is needed to apply pressure up to a certain level at one time and then to precisely adjust the application of pressure immediately before a set level.

As shown in FIG. 4C, when the pusher 11 is pushed in a state that the lever 18 is pressed, it is possible to apply pressure up to a certain level by a force of an operator. As shown in FIG. 4D, when the pusher 11 is rotated in a state that the lever 18 is released, it is possible to precisely adjust the application of pressure based on the engagement of screws.

When the lever 18 is pressed at the time when the balloon is expanded up to a certain volume as a proper pressure is applied, the compression applied by the pusher 11 is fast released, and the pusher 11 slightly moves backwards. A certain space is formed in the space as the balloon is expanded. It is checked whether the fallen spines are recovered to the normal state through the X-ray test. The air pocket is removed, and specially-made liquid bone cement is stably injected into the spines recovered to the normal states for thereby finishing operation.

As described above, in the recovery device according to the present invention, it is possible to properly hold and combine a balloon, a cylinder and a compressor using a box shape body made of a synthetic resin, so that the weight, volume and size can be decreased. The entire construction of a recovery device may be simplified. An assuming process is easy. The manufacture cost can be decreased since it is made in a disposable type.

In addition, a compression releasing operation is easy by using an elastic lever type locking unit and with a simple operation of a lever, so that a gripping operation is easy, and handling a recovery device is easy for thereby implementing a comfortable operation.

There are provided a LCD lamp display at a pressure gauge for an easier check, and an alarm generation unit for generating an alarm signal when a certain pressure is exceeded, so that it is possible to obtain a good visibility for thereby preventing a medical accident.

As the present invention may be embodied in several forms without departing from the spirit or essential characteristics thereof, it should also be understood that the above-described examples are not limited by any of the details of the foregoing description, unless otherwise specified, but rather should be construed broadly within its spirit and scope as defined in the appended claims, and therefore all changes and modifications that fall within the meets and bounds of the claims, or equivalences of such meets and bounds are therefore intended to be embraced by the appended claims.

What is claimed is:

1. In a recovery device for a balloon kyphoplasty which includes a balloon and a balloon body directly inserted into a spine, a cylinder 10 connected with the balloon body using a tube, and a pusher 11 which is assembled at a rear end of the cylinder 10 for thereby performing a pressurizing operation, a disposable type recovery device for a balloon kyphoplasty, comprising:
a box shape body 13 which is integrally assembled at a rear end of the cylinder 10 and partially receives a front end of the pusher 11 for thereby selectively screw-engaging or disengaging the pusher 11 using a pusher locking part 12, with an intermediate portion of the body 13 being combined with two tube members which are separable in a longitudinal direction, and with said box shape body 13 comprising a cylinder mounting part 14 of a front side for receiving a rear end portion of the cylinder, and a hole structure block mounting part 15 being disposed at a rear side of the cylinder mounting part 14 with a partition being disposed between the same, and with said two tube members being combined with each other and being integrally assembled by outer rings 16a and 16b engaged at the step portions of the front and rear ends.

2. The device of claim 1, wherein said pusher locking unit 12 is provided with a locking block 20 which includes:
a lever 18 which is installed at an outer side of the body 13 and is supported in a pin structure while being elastically supported by a spring 17 and operates like a seesaw; and
a screw part 19 of which one side is pin-engaged at a rear end of the lever 18 and which is movable up and down in the block mounting part 15 and has an inner diameter portion engage with the pusher 11.

3. The device of claim 1, wherein said body 13 further includes a pressure gauge 21 provided in the cylinder 10 for measuring pressure, and the pressure gauge is designed to display a pressure data in a LED lamp display method and includes a buzzer for generating an alarm sound when a certain pressure level is reached when a balloon is expanded.

4. The device of claim 1, wherein said cylinder 10 and said pusher 11 are integrally assembled in such a manner that a front end of the pusher 11 is partially straight inserted into a pad 22 of the cylinder 10, and an engaging pin 23 passes through the inserted portion of the pad 22.
